(19) United States
(12) Patent Application Publication
(10) Pub. No.: US 2025/0273342 A1
(43) Pub. Date: Aug. 28, 2025

(54) Systems and Methods for Detecting Patients Susceptible to Falls and Wounds and Providing Notifications for Preventing or Mitigating Falls and Wounds Incurred by the Susceptible Patients

(71) Applicant: Saiva AI, Inc., Los Altos, CA (US)

(72) Inventor: Gill BEJERANO, Stanford, CA (US)

(21) Appl. No.: 19/066,000

(22) Filed: Feb. 27, 2025

Related U.S. Application Data
(60) Provisional application No. 63/558,890, filed on Feb. 28, 2024.

(51) Int. Cl. G16H 50/30; G16H 10/60; G16H 15/00

(52) U.S. Cl. CPC G16H 50/30; G16H 10/60; G16H 15/00

(57) ABSTRACT

Methods and systems for generating a report that identifies wound and fall risks for patients are disclosed. A method includes obtaining historical patient data via one or more databases communicatively coupled with the computer system. The method includes generating a training set including a subset of the historical patient data, training the injury detection system using the training set, and determining, based on new patient data provided to the injury detection system, a patient's fall risk and/or wound risk. The method further includes generating, based on the patient's fall risk and/or wound risk, a patient report and providing caretakers remote access to the patient report. The patient report presents the patient's fall risk and/or wound risk and includes one or more user interface elements for receiving additional information about the patient.

### CROSS-REFERENCE TO RELATED APPLICATIONS

This application claims priority to U.S. Provisional Application Ser. No. 63/558,890, filed on Feb. 28, 2024, and titled “Systems and Methods for Detecting Patients Susceptible to Falls and Wounds and Providing Notifications for Preventing or Mitigating Falls and Wounds Incurred by the Susceptible Patients,” which is incorporated by reference herein for all purposes.

### TECHNICAL FIELD

The disclosed embodiments relate generally to risk identification and, more specifically, the identification of patients susceptible to falls and/or wounds.

### BACKGROUND

Patients that are bedridden and/or undergo complicated medical procedures are at higher risk of incurring wounds and/or falling. Additionally, elderly patients that suffer from chronic and acute illnesses are at higher risk of incurring wounds and/or falling. At times, patients stay at hospitals, nursing homes, or rehabilitation centers and rely on the care of caretakers to move about. Medical facilities can be short-staffed and not have the resources to supervise each patient. Medical facilities need tools to identify patients that are likely to incur wounds and/or likely to fall if not supervised.

As such, a need exists for identifying patients with the highest likelihood of falling or incurring wounds so that special care can be given to those patients.

### SUMMARY

The methods and systems described herein determine a patient's risk of falling and/or incurring a wound. Specifically, methods and systems described herein determine the chances that a patient may trip, slip, stumble, or fall and incur further injury while in the care of a medical facility. Similarly, the methods and systems described herein determine the chances that a patient may experience ulcers, lacerations, rashes, etc. while in the care of a medical facility. By identifying patients at risk of falling and/or incurring a wound, caretakers are able to provide the appropriate supervision and care to patients to ensure that the patients are able to recover and/or prevent injury to themselves. The methods and systems provide an efficient and cost-effective solution for identifying high-risk patients. The methods and systems described herein improve the performance of hospitals and care provided to patients by assisting in the identification of high-risk patients such that appropriate care can be provided to the identified patients. In some embodiments, the methods and systems described herein generate reports that allow medical practitioners to efficiently input notes, diagnosis, or actions taken while caring for a patient. In some embodiments, input received at the report is used to update the patient's determined risk as well as improve models for assessing new patients' risks. The methods and systems described herein reduce the amount of data required by providing a centralized repository for monitoring patients.

One example method of notifying caretakers of a patient's risk of falling or incurring a wound is provided. The example method includes obtaining historical patient data via one or more databases communicatively coupled with the computer system and generating a training set including a subset of the historical patient data. The training set includes one or more features for training an injury detection system. The method also includes training the injury detection system using the training set. The injury detection system is configured to detect at least a fall risk and/or a wound risk for a patient. The method further includes determining, based on new patient data provided to the injury detection system, a patient's fall risk and/or wound risk, generating, based on the patient's fall risk and/or wound risk, a patient report, and providing caretakers remote access to the patient report. The patient report presents the patient's fall risk and/or wound risk and includes one or more user interface elements for receiving additional information about the patient.

Having provided an overview of a first aspect related to notifying caretakers of a patient's risk of falling or incurring a wound, a second aspect (generally related to updating the report based on caretaker inputs) is now described.

Another example method includes, in response to receiving a user input via the one or more user interface elements (of a generated report), creating updated patient data, and determining, based on the updated patient data provided to the injury detection system, the patient's updated fall risk and/or updated wound risk. The updated patient data includes the new patient data and the information. The method also includes generating, based on the patient's updated fall risk and/or updated wound risk, an updated patient report, and providing a notification to the caretakers. The notification provides the caretakers remote access to the updated patient report. In some embodiments, the updated patient report replaces the patient report.

The features and advantages described in the specification are not necessarily all-inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features.

FIG. 1 illustrates an overview of a patient care system, in accordance with some embodiments.

FIG. 2 illustrates an overview of an injury detection system and a reporting system, in accordance with some embodiments.

FIGS. 3A and 3B illustrate example reports generated based on a patient's determined wound risk and/or fall risk.

FIG. 4 is a block diagram illustrating a server system 140 in accordance with some embodiments.

FIGS. 5A and 5B are flow charts illustrating a method of training and using one or more injury detection models for determining wound risks and/or fall risks for patients, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

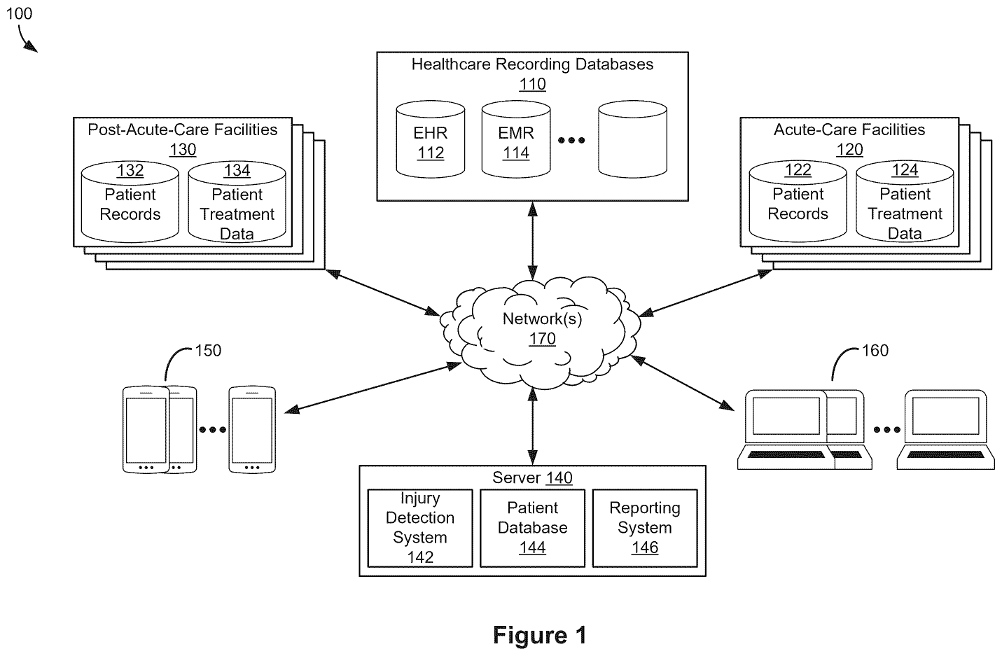

FIG. 1 illustrates an overview of a patient care system, in accordance with some embodiments. The patient care system 100 includes an injury detection system 142 implemented at an electronic device, such as a server 140, that is communicatively coupled with one or more other electronic devices, databases, etc. For example, the patient care system 100 shows the server 140 communicatively coupled with one or more healthcare recording databases 110, one or more acute-care facilities 120, one or more post-acute-care facilities 130, one or more mobile devices 150 (e.g., a smartphone, tablet, smartwatch, etc.), and one or more computers 160 (e.g., desktops, laptops, etc.), each of which can be communicatively coupled via a network 170. The one or more networks 170 can include public communication networks, private communication networks, or a combination of both public and private communication networks. For example, the one or more networks 170 can be any network (or combination of networks) such as the Internet, other wide area networks (WANs), local area networks (LANs), virtual private networks (VPNs), metropolitan area networks (MANs), peer-to-peer networks, and/or ad hoc connections.

The one or more healthcare recording databases 110 include one or more databases storing historical patient healthcare data and current patient healthcare data (e.g., recent healthcare data provided via participating parties (e.g., the acute-care facilities 120, the post-acute-care facilities 130, or other health agencies and/or medical practitioners). The healthcare recording databases 110 can be centralized or distributed. In some embodiments, the healthcare recording databases 110 are updated on a regular basis (e.g., daily, twice a week, weekly). The healthcare recording databases 110 can receive patient data from acute-care facilities 120, post-acute-care facilities 130, the server system 140, and/or other communicatively coupled devices to update the patient data. In this way, the healthcare recording databases 110 provide a centralized repository of patient data that is kept up to date and allows for easy distribution of patient data. In some embodiments, the healthcare recording databases 110 include electronic health records (EHRs 112), electronic medical records (EMRs 114), and/or other similar recordkeeping services.

In some embodiments, the healthcare recording databases 110 include data relating to medical history, medical conditions, medical diagnosis, medication and allergies, immunization status, laboratory tests ordered, laboratory test results, radiology images, vital signs, frequency of visits to an acute-care facility and/or a post-acute-care facility, number of times admitted to an acute-care facility and/or a post-acute-care facility, length of stay at an acute-care facility and/or a post-acute-care facility, notes taken during treatment (e.g., notes from a physician, therapist, nurse, and/or other medical practitioner), free form progress notes, diet information, and other patient assessment data. In some embodiments, the healthcare recording databases 110 include patient demographic information, personal statistics (e.g., age, weight, etc.), social visitor data, billing information, socio-economic information, and other patient-specific data. In some embodiments, the healthcare recording databases 110 include patient data collected via wearable devices. For instance, wearable devices, such as smartwatches, and fitness trackers, mobile devices, pedometers, etc. may collect sleep data, movement data, heart rate, stress levels, etc. that are stored in healthcare recording databases 110.

In some embodiments, the healthcare recording databases 110 include environmental data such as weather, air quality, water quality, pollution, etc. In some embodiments, healthcare recording databases 110 include data received from other external systems or sources. For example, the healthcare recording databases 110 may include data from one or more agencies (e.g., the Centers for Disease Control and Prevention (CDC), Environmental Protection Agency (EPA), Food and Drug Administration (FDA), fire departments, or other agencies), local government, media outlets, Internet sources, etc. that can be relevant in determining patients' risk at a particular point in time. For instance, unusual air pollution due to fire could be provided by a fire department, viruses spread by food could be provided by a government agency, and natural or man-made hazards (e.g., radiation) could be communicated by media outlets, etc. In some embodiments, the healthcare recording databases 110 include data that reflects any readmissions of a patient from one or more post-acute-care facilities to one or more acute-care facilities.

The above examples are non-limiting. The healthcare recording databases 110 are configured to store any data that could affect a patient's health.

The healthcare recording databases 110 provide historical and/or current patient data to the server system 140 for training a machine-learning system and/or using a trained machine-learning system for determining and/or detecting patients' risks of incurring an injury (e.g., a wound and/or falling), as discussed below. In some embodiments, the healthcare recording databases 110 provide data to the server system 140 periodically (e.g., twice a day, daily, weekly, etc.). In some embodiments, the healthcare recording databases 110 provide data to the server system 140 responsive to a request from the server system 140.

In some embodiments, the one or more acute-care facilities 120 include emergency rooms, surgical centers, intensive care units, detoxification units, neonatal intensive care units (NICUs), emergency psychiatric services, hospitals, hospital emergency departments, ambulatory surgery centers, urgent care centers, or other short-term stay facilities. Acute-care facilities 120 provide care during which a patient is treated for severe injury or illness, trauma, urgent medical condition, or during recovery from surgery. Acute care may require a patient to stay at a facility; however, patients in acute-care facilities 120 are generally discharged to post-acute-care facilities 130 to complete their recovery as soon as they are deemed healthy and stable. In some embodiments, acute-care facilities 120 collect patient data for one or more patients including demographics, medical history, medications and allergies, immunization status, diagnosis, laboratory test results, radiology images, vital signs, personal statistics (e.g., age, weight, etc.), and billing information (e.g., patient records 122). The acute-care facilities 120 can collect treatment, notes, medical analysis, adjustments to medications or treatments, and/or other actions and recommendations performed by medical practitioners (e.g., physicians, registered nurses (RNs), etc.), which can be stored in the patient records 122 and/or in patient treatment data 124. The acute-care facilities 120 can collect data analogous to the data described above in reference to the healthcare recording databases 110. In some embodiments, the acute-care facilities 120 provide their collected data to the healthcare recording databases 110, the post-acute-care facilities 130, the server 140, and/or communicatively coupled devices via networks 170.

In some embodiments, the one or more post-acute-care facilities 130 include nursing homes, dialysis centers, physician offices, rehabilitation facilities, and psychiatric institutions. Post-acute-care facilities 130 provide continued medical treatment to patients discharged from an acute-care facility 120. Post-acute-care facilities 130 emphasize recovery, recuperation, rehabilitation, and symptom management. For example, a patient recovering from a stroke often requires rehabilitative therapies to help them fully recover or prevent them from returning to an acute-care facility 120. Post-acute-care facility 130 services range from intensive short-term treatment to long-term care. As such, the goal of post-acute-care facilities 130 is to ensure that patients do not return to acute-care facilities.

In some embodiments, the post-acute-care facilities 130 collect patient data for one or more patients as described above with respect to acute-care facilities 120. The patient data collected at the post-acute-care facilities 130 is collected during the continuous treatment of the patient while at the post-acute-care facility 130 and stored in patient records 132 and patient treatment data. As such, the patient data collected at the post-acute-care facilities 130 could be more up-to-date than the patient data collected at the acute-care facilities 120. In some embodiments, the patient data collected at the post-acute-care facilities 130 is analogous to the patient data collected at the acute-care facilities 120 and/or stored in the healthcare recording databases 110. In some embodiments, the post-acute-care facilities 130 provide their collected data to the healthcare recording databases 110, the acute-care facilities 120, the server 140, and/or communicatively coupled devices via networks 170.

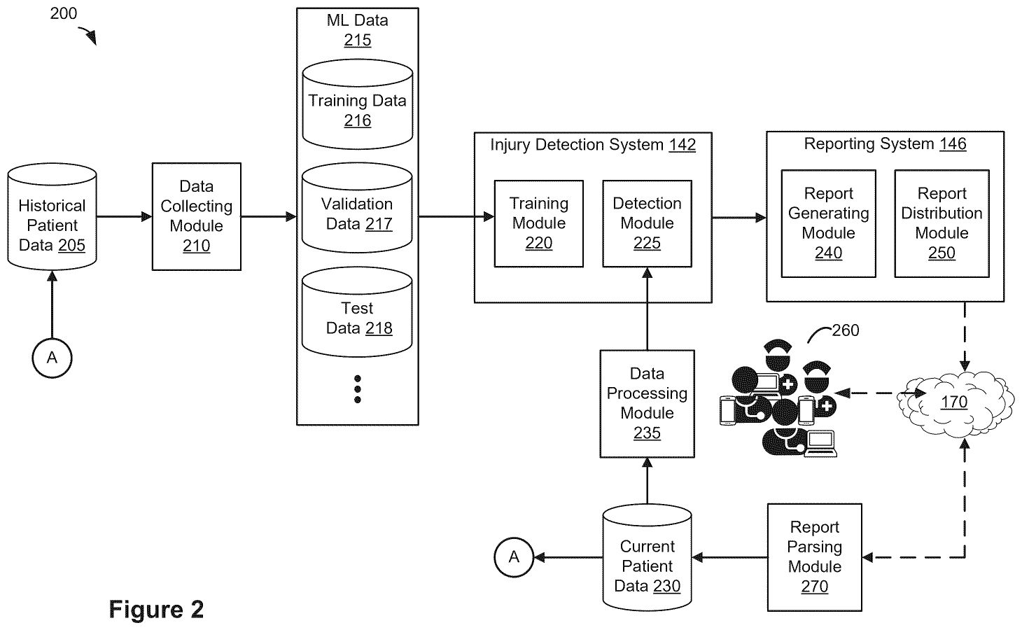

The server system 140 can include the injury detection system 142, a patient database 144, and a reporting system 146. The server system 140 is configured to receive historical patient data and/or current patient data from the healthcare recording databases 110, the acute-care facilities 120, the post-acute-care facilities 130, the mobile devices 150, and the computers 160. The server system 140 stores the patient data received by the healthcare recording databases 110, the acute-care facilities 120, the post-acute-care facilities 130, the mobile devices 150, and/or the computers 160 in patient database 144. The patient data within the patient database 144 is processed to remove any personal identifiable information and/or otherwise anonymize the data to protect patient information. The patient data received from the healthcare recording databases 110, the acute-care facilities 120, the post-acute-care facilities 130, the mobile devices 150, and the computers 160 and/or the patient data stored within the patient database 144 is processed (e.g., by a data collecting module 210 and/or data processing module 235; FIG. 2) before it is provided to the injury detection system 142, as discussed below.

The injury detection system 142 is configured to use patient data received from the healthcare recording databases 110, the acute-care facilities 120, the post-acute-care facilities 130, the mobile devices 150, and the computers 160 and/or the patient data stored within the patient database 144 for training one or more machine-learning systems and/or models. In particular, the injury detection system 142 uses at least a portion of processed patient data (e.g., which can include training data 216, validation data 217, test data 218, and other data described below in reference to FIG. 2) to train the one or more machine-learning systems and/or models. Trained machine-learning systems and/or models can be stored at the injury detection system 142 and/or a communicatively coupled database (e.g., server database 140). The injury detection system 142, after training one or more machine-learning systems and/or models, is configured to determine patients' risks of incurring an injury, such as a wound (e.g., a contusion, a pressure ulcer, a lesion, an abrasion, etc.) and/or a fall. For example, a post-acute-care facility 130 can provide the injury detection system 142, via the server 140, patient data related to a new patient to receive information about the new patient's risk of incurring an injury.

The server 140 can generate a patient report based on patients' risks of incurring an injury. The patient report can be generated by the reporting system 146, which receives the determined risk of incurring an injury from the injury detection system 142. The reporting system 146 generates patient reports that can be presented at different devices. The patient report, when generated, can be provided to one or more acute-care facilities 120, one or more post-acute-care facilities 130, one or more mobile devices 150, one or more computers 160, and/or other communicatively coupled devices. Alternatively, or in addition, the generated patient report is provided to medical practitioners and/or other caretakers.

The medical practitioners and/or other caretakers are provided remote access to the patient report such that the medical practitioners and/or other caretakers can review the patient's fall risk and/or wound risk. For example, the patient report can be accessed via an application (e.g., mobile application, web-based application, and/or program running on a computer), a web browser, web-based platforms, secured networks, etc. Additionally, the patient report allows the medical practitioners and/or other caretakers to provide user input including information about the patient, such as medications, treatments, symptoms, conversations, environment conditions and/or changes in the environment, and/or other factors relevant to the patient's care. The user input can be in a structured format (e.g., options from a drop-down menu, radio buttons, check boxes, etc.) and/or unstructured format (e.g., text, handwriting, etc.).

In some embodiments, information provided on the patient report (e.g., via a user input) is provided to the server 140, and the server 140 updates the patient data based on the user inputs. Before the patient data is updated, the information is formatted to be consistent with the existing patient data. For example, handwriting can be parsed to determine relevant factors within the handwriting and reformatted to be included in the patient data. The updated patient data is processed and provided to the injury detection system 142, which in turn determines an updated fall risk and/or updated wound risk. When the fall risk and/or the wound risk are updated, the reporting system 146 generates an updated patient report and provides a notification to the medical practitioners and/or other caretakers. The notification provides medical practitioners and/or other caretakers remote access to the updated patient report (which replaces the previous patient report).

In some embodiments, the patient reports are configured to be accessed locally at the acute-care facilities 120, post-acute-care facilities 130, mobile devices 150, computers 150, and/or other communicatively coupled devices. For example, the server 140 can allow the patient reports to be stored locally at a respective location (e.g., at a client database, computer, or other device). In some embodiments, the patient reports are stored at the reporting system 146 and/or other communicatively coupled database.

The mobile devices 150 and the computers 160 can be associated with the healthcare recording databases 110, the acute-care facilities 120, the post-acute-care facilities 130, and/or the server 140. The mobile devices 150 and the computers 160 can include one or more programs or applications for displaying information provided by the healthcare recording databases 110, acute-care facilities 120, post-acute-care facilities 130, and/or server 140. In some embodiments, the mobile devices 150 and the computers 160 include one or more inputs and/or outputs for interacting with the information provided. For example, the mobile devices 150 and the computers 160 can include a display, a keyboard, a touch screen, a mouse, a microphone, an audio output, and/or other devices.

Although the patient care system 100 shows the injury detection system 142 and the reporting system 146 implemented at a server 140, the injury detection system 142 and the reporting system 146 can be implemented on other devices. For example, the injury detection system 142 and/or the reporting system 146 can be implemented at a mobile device 150, a computer 160, an acute-care facility 120, a post-acute-care facility 130, etc. In some embodiments, the injury detection system 142 and/or the reporting system 146 are implemented at more than one server 140 and/or communicatively coupled device. In some embodiments, the injury detection system 142 and/or the reporting system 146 are implemented at any combination of healthcare recording databases 110, acute-care facilities 120, post-acute-care facilities 130, servers 140, mobile devices 150, computers 160, etc.

FIG. 2 illustrates an overview of an injury detection system and a reporting system, in accordance with some embodiments. Overview 200 shows a detailed process of training the injury detection system 142 and generating and distributing one or more reports via the reporting system 146. Overview 200 includes historical patient data 205, a data collecting module 210, machine-learning data 215, the injury detection system 142, the reporting system 146, current patient data 230, a report parsing module 270, and a data processing module 235, each of which can be included in a server 140 and/or in an electronic device communicatively coupled with a server 140 or other device described above in reference to FIG. 1.

As shown in overview 200, the historical patient data 205 is provided to a data collecting module 210. The historical patient data 205 is previously stored patient data that is provided from one or more healthcare recording databases 110, acute-care facilities 120, post-acute-care facilities 130, servers 140 (e.g., stored in patient database 144), mobile devices 150, computers 160, and/or other devices or sources described above in reference to FIG. 1. The data collecting module 210 is configured to process the historical patient data 205 and determine one or more features for training, validating, and/or testing a machine-learning system. More specifically, the data collecting module 210 identifies portions of the historical patient data 205 for training an injury detection model (e.g., a wound and/or fall risk detection module). In some embodiments, the data collecting module 210 identifies portions of the historical patient data 205 between a predetermined time period and/or a predetermined number of patients from the historical patient data 205. The predetermined time period for the historical patient data 205 can span days, weeks, months, years, etc.; or any other time period defined by a user. The predetermined number of patients from the historical patient data 205 can include tens, hundreds, thousands, millions, etc. of patients; or a number of patients defined by the user.

As described above, the one or more features determined by the data collecting module 210 are used for training an injury detection model. The features are selected such that an injury detection model can determine a patient's risk of falling (e.g., slipping, tripping, stumbling, etc.) and/or incurring a wound (e.g., lacerations, sores, ulcers, contusions, abrasions, etc.). Such features can include a patient's demographic information (e.g., age, sex, etc.), illnesses, diseases, sicknesses, conditions, and/or other potential ailments, as well as the patient's location (e.g., home, hospital, nursing home, etc.), provided care, hygiene, activity (e.g., sedentary, light physical activity, moderate physical activity, etc.), and environmental conditions (e.g., type of floor, obstacles, foot traffic, weather, etc.). The above examples are non-limiting, and more or fewer features can be used for training an injury detection model. In some embodiments, the data collecting module 210 determines one or more features based on geographic location (e.g., accessibility of a house, neighborhood, building, and/or other areas a patient frequents). In some embodiments, the data collecting module 210 determines one or more features based on a combination of different portions of the historical patient data 205. For example, the data collecting module 210 can combine historical weather patterns with the walkability of a neighborhood that is used to determine a patient's risk of falling while going for a walk.

In some embodiments, features used by the injury detection model for determining a patient's risk of falling and/or incurring a wound include a patient's age, falls and wounds history, admitted location (e.g., location from which the user was admitted), days since first admission, days since last arrival, transfer reason, progress note types (e.g., quarterly recreation notes, eMAR-medication administration note, etc.), patient assessments (e.g., eINTERACT change in condition evaluation), progress notes text stream, pain level, vaccination record, treatment and medication orders, day of the week, and month of the year. As described above, received patient data can be processed and/or formatted to be used by the injury detection model. In particular, patient data can be received in different mediums and/or in different formats and be processed and/or formatted into one or more features that can be input into the injury detection model. The above-mentioned features are non-limiting, and more or fewer features than those referenced above can be used to determine a patient's risk of falling and/or incurring a wound.

Features are a transformation of patient data from a raw state (e.g., unstructured) to a state suitable (e.g., structured) for generating one or more injury detection models. Additionally, features are inputs to one or more injury detection models. The data collecting module 210 transforms each element of the historical patient data 205 into one or more features. In some embodiments, the data collecting module 210 transforms a subset of and/or a combination of the historical patient data 205 elements into one or more features. For example, the data collecting module 210 can generate one or more features for a number of days that a patient has been bedridden; a patient's injury, sickness, or disease; each medication that a patient is taking or has taken; a number of dosages of the medication that a patient has been given or refused to take; and/or any other data points in the historical patient data 205. In some embodiments, the features can define minimums and maximums for occurrences in the historical patient data 205. For example, the data collecting module 210 can generate one or more features for a patient's maximum and/or minimum daily blood pressure. The features can be combinations of related or unrelated data points in the historical patient data 205. In short, any data element or different combination of data elements in the historical patient data 205 described in reference to FIG. 1 can be used to determine one or more features.

As described below, the one or more features generated by the data collecting module 210 are provided to a training module 220 (e.g., as training data 216, validation data 217, and/or test data 218) to define and/or structure the inputs of an injury detection model. In some embodiments, the training module 220 applies different techniques (e.g., algorithms) to train one or more injury detection models that will make determinations (e.g., scores, evaluations, and/or predictions) based on the provided features.

In some embodiments, the data collecting module 210 is configured to clean, standardize, sanitize, sample, normalize, and/or organize the historical patient data 205 before or in conjunction with determining one or more features. Alternatively, in some embodiments, the data collecting module 210 receives the historical patient data 205 after the historical patient data 205 has been cleaned, standardized, sanitized, sampled, normalized, and/or organized. In some embodiments, the data collecting module 210 performs optical character recognition (OCR) on different notes (handwritten or typed) to determine information that can be used to train an injury detection model. In some embodiments, the data collecting module 210 does not process the patient data and uses the raw information as stored in the historical patient data 205.

The data collecting module 210 is configured to provide machine-learning data 215 for training an injury detection model. The machine-learning data 215 includes one or more subsets of data for training, validating, and testing an injury detection model. For example, the machine-learning data 215 includes at least training data 216, validation data 217, and test data 218. Each subset of machine-learning data 215 is based on the one or more features for training an injury detection model (as determined by the data collecting module 210).

The training data 216 is a subset of the machine-learning data 215 that is used by the training module 220 of the injury detection system 142 to train one or more injury detection models. The validation data 217 is a subset of the machine-learning data 215 that is held back from training the one or more injury detection models. The validation data 217 is used by the training module 220 to evaluate performance of the injury detection models (e.g., accuracy rate) while tuning hyperparameters (e.g., number of hidden units, number of layers, etc.) of the injury detection models. Because the validation data 217 is used to evaluate the performance of the injury detection models while tuning the hyperparameters of the injury detection models, the validation data 217 becomes biased over time. An unbiased set of data, the test data 218, is used by the training module 220 to evaluate the performance of fully trained injury detection models. Specifically, the test data 218 is a subset of the machine-learning data 215 that is held back from training the one or more injury detection models and is distinct from the training data 216 and the validation data 217. The training, validation, and testing of the one or more injury detection models is discussed below in reference to the training module 220.

Turning to the injury detection system 142, received machine-learning data 215 is used to train and enable one or more injury detection models. Specifically, the training data 216, the validation data 217, and the testing data 218 are used by the training module 220 to generate one or more injury detection models. In some embodiments, the training module 220 trains one or more injury detection models based on unsupervised learning, supervised learning, semi-supervised learning, deep learning, and/or any other type of neural network. The training module 220 uses the training data 216 to determine one or more parameters and hyperparameters for an injury detection model. A parameter is a variable that is internal to the injury detection model and whose value can be estimated from the training data 216. Each parameter is learned and not manually set by a user. The one or more parameters determined during training of an injury detection model are saved as part of the injury detection model and used by the injury detection model to determine a wound risk and/or fall risk for a patient. The wound risk and/or fall risk, for purposes of this disclosure, are an evaluation, score, and/or probability of a patient incurring a wound or experiencing a fall. The performance (e.g., accuracy) of a trained injury detection model is based on the determined parameters of the trained injury detection module.

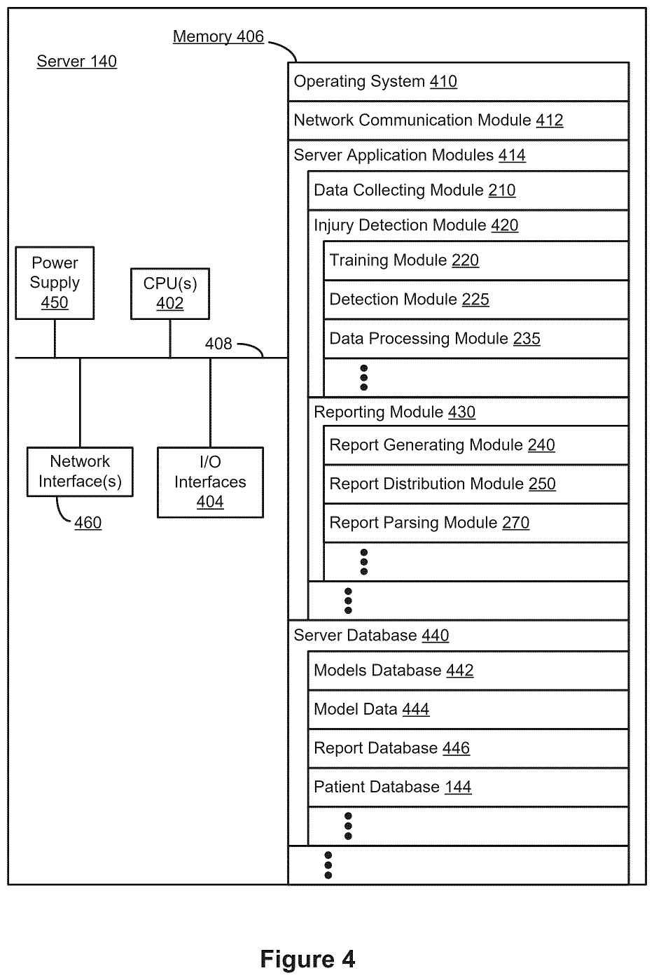

Hyperparameters, on the other hand, are external to the injury detection model and include values that are not estimated from the training data 216. In some embodiments, one or more hyperparameters are used in the training process to help estimate the one or more parameters. The one or more hyperparameters are defined by a user (e.g., via the I/O interfaces 404 or network interfaces 460; FIG. 4). Alternatively, or in addition, in some embodiments, the one or more hyperparameters are set using heuristics. As discussed below, the training module 220 uses the validation data 217 to determine (e.g., tune) one or more hyperparameters for an injury detection model.

In some embodiments, the training module 220 generates injury detection models that include one or more initial hyperparameters that are configurable (e.g., can be modified by a user as described above). The validation data 217 is input into different trained injury detection models such that a user can adjust the hyperparameters of an injury detection model. The one or more adjustments to the hyperparameters are used to improve the performance of the injury detection model. In some embodiments, one or more injury detection models can have the same or different hyperparameters.

In some embodiments, after an injury detection model is tuned, the training module 220 uses the test data 218 to determine the performance of the injury detection model. The test data 218 is used with a trained injury detection model to determine one or more wound risks and/or fall risks. The performance of a trained injury detection model is based on the accuracy of the wound risks and/or fall risks for patients in the test data 218 (e.g., which include known values). In some embodiments, injury detection models with a performance (e.g., accuracy rate) at or above a predetermined accuracy rate (e.g., at least 85 percent accurate) are stored to a models database 442 (FIG. 4). Injury detection models with a performance below the predetermined accuracy rate are not stored to the model database 442. In some embodiment, the injury detection models are stored in the model database 442 and include the parameters, hyperparameters, and/or other model weights for using the injury detection model on new patient data.

In some embodiments, the training module 220 is configured to use previously generated injury detection models (stored and/or discarded) to train new injury detection models. In some embodiments, the previously generated injury detection models can be used to determine new features and/or adjust previously determined features of the machine-learning data 215. For example, the machine-learning data 215 can be updated to include new features, different combination of features, using more or fewer features, modifying the previously selected features, and/or other adjustments. In this way, injury detection models trained by the training module 220 are continuously improved with the information learned over repeated training.

Injury detection models with a performance at or above the predetermined accuracy rate are used by a detection module 225 of the injury detection system 142 to determine a wound risk and/or a fall risk for a patient. Specifically, the detection module 225 receives and uses current patient data 230 to determine a wound risk and/or a fall risk for a patient. The current patient data 230 includes recently acquired patient data that is similar to the data stored in the historical patient data 205. The current patient data 230 is processed by the data processing module 235 before it is provided to the detection module 225. In some embodiments, the data processing module 235 is part of the injury detection system 142. The data processing module 235 is analogous to the data collecting module 210 and determines one or more features based on the current patient data 230 that are provided to the detection module 225 for use with one or more injury detection models. The detection module 225 formats, cleans, standardizes, sanitizes, samples, normalizes, and/or organizes the current patient data 230 such that the inputs provided to the detection module 225 are specific to a particular injury detection model. Specifically, the features (based on the historical patient data 205) provided to the training module 220 define the inputs of a particular injury detection model and, as such, the data processing module 235 generates analogous features, based on the current patient data 230, such that the inputs remain consistent (e.g., the one or more features determined by the data processing module 235 are the same features used to train a respective injury detection model).

The detection module 225 can use one or more injury detection models to determine wound risks and/or fall risks for patients. The detection module 225 can use the one or more injury detection models individually, in parallel, or as an ensemble (e.g., sequentially or in a combination with each other). The detection module 225 can use the one or more injury detection models in parallel to eliminate conflicting and/or abnormal results and/or verify results generated by one or more injury detection models. Alternatively, or in addition, detection module 225 can use the one or more injury detection models as an ensemble to isolate particular results (e.g., a first injury detection model to identify fall risks and a second injury detection model to identify wound risks). In some embodiments, the detection module 225 selects one or more injury detection models for determining wound risks and/or fall risks based on the accuracy of the injury detection models.

As described above, the wound risk and/or fall risk (e.g., as determined by the detection module 225) includes an evaluation, score, and/or probability of a patient incurring a wound or experiencing a fall. The wound risks and/or fall risks determined by the detection module 225 can identify one or more features resulting in a particular wound risk and/or fall risk, as well as one or more features that can result in a change to a particular wound risk and/or fall risk. For example, a fall risk can indicate that a patient with a recent hip replacement has a high probability of falling and that the fall risk can be increased due to weather conditions (e.g., rain) and decreased with prescribed medical equipment (e.g., a walker). In some embodiments, each feature that contributes to, mitigates, and/or prevents a wound risk and/or fall risk is evaluated, scored, and/or assigned a probability. For example, a bedridden patient with a high wound risk can have a feature associated with activity level (e.g., sedentary lifestyle) assigned with a high score (e.g., a key contributor to the wound risk) and a feature associated with patient repositioning (e.g., turning a patient on their side, back, etc.) assigned with a moderate score (e.g., a mitigator to the wound risk).

The injury detection system 142 provides determined wound risks and/or fall risks for patients to the reporting system 146. The reporting system 146 includes a report generating module 240 and a report distribution module 250. The report generating module 240 generates one or more reports corresponding to patients for whom wound risks and/or fall risks were determined. Specifically, the report generating module 240 generates the reports based on inputs (e.g., features determined by the data processing module 235) provided to the one or more injury detection models and/or outputs (e.g., determined wound risks and/or fall risks) of the one or more injury detection models used by detection module 225. The generated reports include information corresponding to the patients including each patient's wound risks and/or fall risks and/or other data included in the current patient data 230. For example, generated reports can include patient demographic information, sicknesses, illnesses, diseases, injuries, condition, performed procedures, activity level, geographic location, living conditions, and/or other information identified above with reference to FIG. 1.

In some embodiments, the report generating module 240 is another model trained using the machine-learning data. The report generating module 240 can be a natural language processing model configured to generate a detailed report based on the determined wound risks and/or fall risks. For example, the report generating module 240 can be configured to use the one or more inputs and outputs used by an injury detection model to generate one or more explanations for the determined wound risks and/or fall risks and/or mitigating actions that can be performed to reduce the wound risks and/or fall risks. The report generating module 240 can identify and explain features that contribute to a determined wound risk and/or fall risk, as well as features that mitigate the determined wound risks and/or fall risks and/or can prevent the occurrence of a wound and/or fall. In some embodiments, reports generated by the report generating module 240 include one or more numerical values (e.g., percentages, scores, etc.) for the determined wound risks and/or fall risks and associated features and/or visual data representations (e.g., graphs, plots, tables, pie charts, gauge, etc.) for the determined wound risks and/or fall risks and associated features.

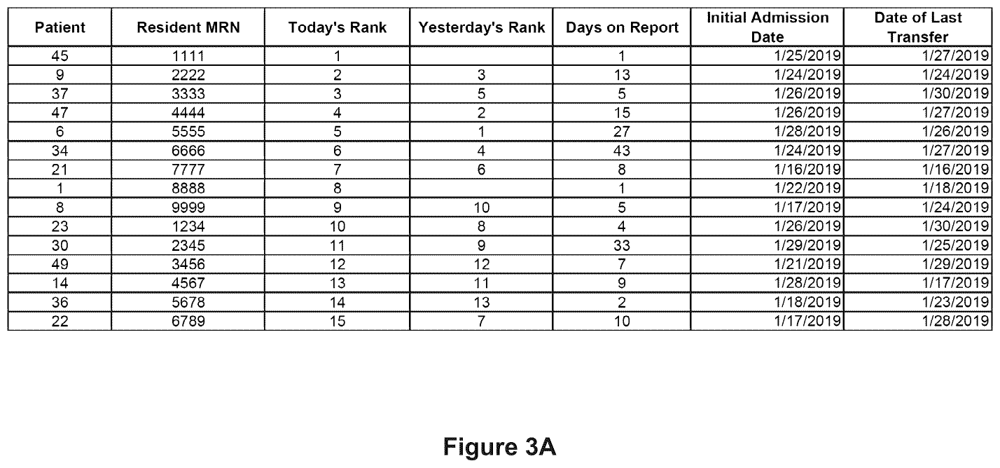

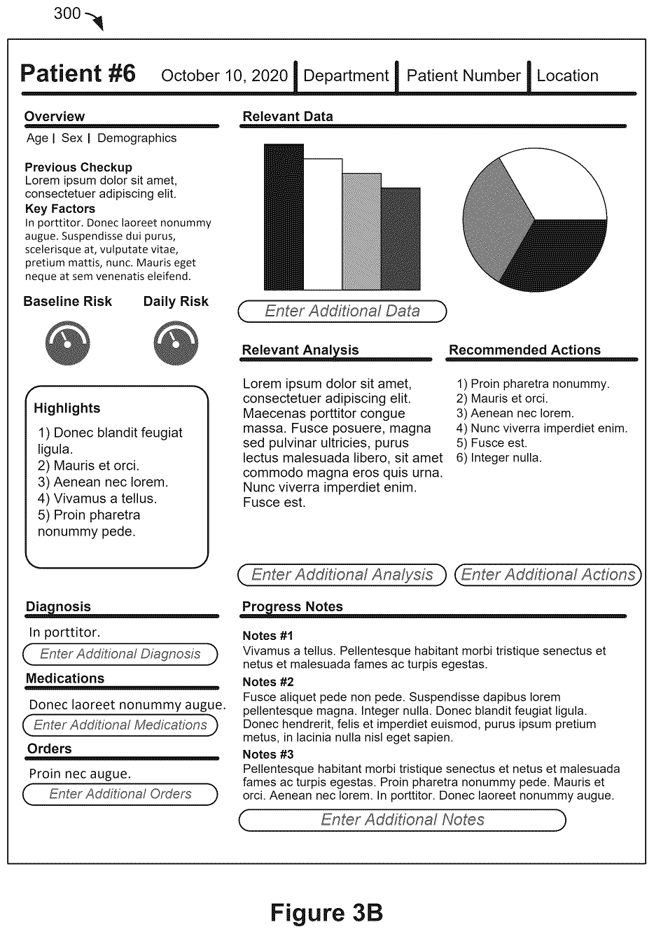

The report generating module 240 is configured to generate the reports in a predetermined format and/or layout. For example, the report generating module 240 can generate a report to include one or more labels, rows, columns, headers, titles, legends, axis, or other characteristics for the presentation of the determined wound risks and/or fall risks and associated features, as well as any other patient information (e.g., demographic information or other data stored within current patient data 230). The report generating module 240 is configured to, based on the determined wound risks and/or fall risks and associated features, include one or more user interface elements in a generated report. The one or more user interface elements allow a caretaker to provide additional information that can be used to update the determined wound risks and/or fall risks. Example reports generated by the report generating module 240 are described below in reference to FIGS. 3A and 3B.

Alternatively, or in addition, in some embodiments, the report generating module 240 formats the patient reports such that relevant information is presented to medical practitioners and/or other caretakers irrespective of their type of device (computer, mobile device, etc.), display size, available computational resources (e.g., available volatile, non-volatile memory, processing power, etc.). For example, the report generating module 240 can compress a patient report and/or modify information in a patient report that is to be shared with a computing device with reduced computational resources (computing devices with fewer computation resources to have full access to the patient reports). In another example, the reporting system 146 can format the patient report such that it can be accessed by web pages and/or applications. In yet another example, the report generating module 240 can format the patient report such that it is scaled for different display sizes and/or includes different tabs or tables for displaying patient information effectively.

The report distribution module 250 is configured to provide access to the reports generated by the report generating module 240. Specifically, the report distribution module 250 makes the generated report accessible to one or more caretakers 260 via a network 170 (FIG. 1). In some embodiments, the report distribution module 250 distributes the generated report to the caretakers 260 via acute-care facilities 120, post-acute-care facilities 130, mobile devices 150, and computers 160 coupled with the server 140 via the network 170 (FIG. 1). In some embodiments, the report distribution module 250 provides the generated report to the caretakers 260 via a patient portal that is in communication with the server 140. In some embodiments, the reports provided via the report distribution module 250 are communicatively coupled with the injury detection system 142 and/or the reporting system 146 via network 170 such that inputs provided by the caretakers 260 can be used to update the determined wound risks and/or fall risks using the one or more injury detection models. Alternatively, or in addition, in some embodiments, the reports distributed using the report distribution module 250 can be updated in response to inputs provided by the caretakers 260 (e.g., the report is updated without requiring the injury detection models to be run a subsequent time). For example, a caretaker 260 including a particular action performed on a patient in a generated report can cause the report to be updated to show updated and/or new wound risks and/or fall risks. In some embodiments, the report is updated in real time, in substantial real time (e.g., within minutes), or within the day.

The inputs provided by the caretakers 260 can be handwritten inputs, electronic inputs (e.g., typed descriptions), uploaded files (e.g., medical reports, studies, pictures, videos, etc.), etc. As discussed in detail below, the inputs provided by the caretakers 260 can be processed by a report parsing module 270 (which works in conjunction with the report distribution module 250) to determine relevant information for updating the determined wound risks and/or fall risks.

In some embodiments, the report distribution module 250 is further configured to provide one or more notifications and/or alerts to the caretaker 260. In some embodiments, the report distribution module 250 provides the notifications and/or alerts when a new and/or updated report is provided. In some embodiments, the report distribution module 250 provides the notifications and/or alerts when an input is received at a report. For example, a first caretaker supervising a patient can perform an action for the patient (e.g., take the patient to physical therapy) and a second caretaker supervising the patient can be notified of the first caretaker's action. The report distribution module 250 can also provide notifications and/or alerts in response to a triggering event. A triggering event can be one or more predetermined events or caretaker defined events. For example, a triggering event can be a determined wound risk and/or fall risk for a patient above a predetermined level (e.g., above 70 percent), a recent medication provided to the patient, the last checkup performed on the patient, an elapsed predetermined time interval (e.g., three hours), etc. The above examples are non-limiting, and any type of triggering event can be defined for providing notifications and/or alerts to a caretaker 260.

Returning to overview 200, inputs received via the reports distributed by the report distribution module 250 are provided to the report parsing module 270. The report parsing module 270 can be part of the reporting system 146. In some embodiments, the report parsing module 270 works in conjunction with the report distribution module 250. Specifically, the report parsing module 270 can analyze one or more inputs provided by caretakers 260 while the report distribution module 250 makes the reports available. The report parsing module 270 can be used to detect and extract inputs provided by the caretakers 260. The inputs extracted using the report parsing module 270 can be used to update the reports provided by the report distribution module 250. For example, a caretaker 260 can summarize a patient's condition and the report parsing module 270 can identify relevant portions of the summary to update the report in real time, in substantial real time, or within the day.

In some embodiments, the report parsing module 270 is configured to process the inputs provided by the caretakers 260. Specifically, the report parsing module 270 is configured to clean, standardize, normalize, organize, etc. the reports edited by the caretakers 260 and/or any inputs provided by the caretakers 260. In some embodiments, the report parsing module 270 performs OCR on the inputs or the reports to extract relevant information. The report parsing module 270 provides the processed data to the current patient data 230 to update the current data. The updated current patient data 230 can be used by the one or more injury detection models, as discussed above, to update or determine new wound risks and/or fall risks for patients. Additionally, as described above, in some embodiments, the data processed by the report parsing module 270 is provided to update the report. The report parsing module 270 allows for patient information to be updated regardless of the format in which the input is received (e.g., handwritten notes, structured data, etc.) and/or platforms used (e.g., web application, host site, device application, etc.). The report parsing module 270 can convert received inputs into a standardized format, store the inputs, and/or provide the inputs for subsequent updates as described herein. In some embodiments, the report parsing module 270 allows for updates to patient information to be shared in real time (e.g., via one or more messages and/or notifications) and/or used in the generation of updated patient reports.

In some embodiments, the updated current patient data 230 is provided to the historical patient data 205. In this way, the historical patient data 205 can be updated with any new information regarding patients, and future models can be trained to further improve accuracy.

FIGS. 3A and 3B illustrate example reports generated based on a patient's determined wound risk and/or fall risk. FIG. 3A shows a listing of one or more patients and the patient's respective rank (based on likelihood of incurring a wound risk and/or experiencing a fall). FIG. 3B illustrates a report generated for a particular patient.

As shown in FIG. 3A, the listing of one or more patients includes patient identifying information, current patient rank, and other patient information. For example, patient 45 has an identifying number “1111,” has the highest risk of incurring a wound or experiencing a fall (as denoted by rank 1), and has only been on the report one day. In some embodiments, the patient information is anonymized to protect patient privacy. The listing of one or more patients can include a predetermined number of patients (e.g., 10 patients, 50 patients, 100 patients), patients that satisfy a risk threshold (e.g., above 50 percent risk, 60 percent risk, 70 percent risk), recent patients, and/or recurring patients. Caretakers can reorganize and/or adjust the listing as required. For example, caretakers can provide an input to change the order from highest rank to lowest rank for the day; by date of intake; days on the list; etc. Each patient on the listing of one or more patients is associated with a respective detailed report (as shown in FIG. 3B).

FIG. 3B illustrates an example detailed report for a particular patient, in accordance with some embodiments. The detailed report 300 includes, for display, collected patient data. In particular, in some embodiments, the detailed report 300 includes ranking information and/or patient information as described above with reference to FIG. 2. As described above in reference to FIG. 2, the report can be generated using a natural language processing model that uses the determined wound and/or fall risk and associated features for generating one or more explanations, summaries, descriptions, etc. for the determined wound and/or fall risk. In some embodiments, the detailed report for a patient is generated and displayed in response to selection of the patient's name and/or information in a patient listing or ranking shown in FIG. 3A.

The detailed report 300 includes the patient's name (or pseudonym to protect the patient's privacy), patient identifier (or number), patient location (e.g., facility, room number, bed number, etc.), and report date. The detailed report 300 includes one or more sections, including an overview section, relevant data section, relevant analysis section, risks section, highlights section, diagnosis section, medications section, orders section, progress notes section, and recommended actions section. The above-example sections are non-exhaustive and additional sections not shown can be included.

The overview section provides a snapshot (or summary) of the patient's information. In some embodiments, the overview section includes the patient's age, sex, and/or race. In some embodiments, the overview section includes the patient's length of stay (LoS), such as the number of hours, days, months, etc. that the patient has been at the medical facility (e.g., post-acute-care facility). In some embodiments, the overview section includes information regarding a determined wound and/or fall risk for a patient. The overview section can include a summary of the determined wound and/or fall risk as well as an evaluation of the determined wound and/or fall risk (e.g., has improved over time, has stagnated, changes in environment have worsened the risk, etc.). The overview section can also identify key factors (e.g., features as described above in reference to FIG. 2) that contribute to the patient's determined wound and/or fall risk. In some embodiments, the overview section includes the patient's medical history (e.g., chronic, or acute conditions or illnesses, diseases, mental conditions, disorders, etc.).

The relevant data section includes patient data related to the patient's determined wound and/or fall risk. For example, the relevant data section can include information on the user's demographic information, weight, vitals (e.g., blood pressure, weight, oxygen saturation, pulse, etc.), geographic information, and/or any features that are used by injury detection model for determining the patient's wound and/or fall risk. In some embodiments, the relevant data section includes one or more data visualizations for the patient data (e.g., data fields, spreadsheets, tables, graphs, charts, plots, fishbone diagrams, etc.). In some embodiments, the data visualizations of the vitals section 804 include data over a predetermined period of time (e.g., 10 days, 14 days, 30 days, 80 days, etc.). In some embodiments, the data visualizations include one or more markers such as min/max thresholds, time windows (e.g., two weeks ago), etc.

As shown by the detailed report 300, the relevant data section includes a user interface element to allow a caretaker to provide additional data. The additional data can be handwritten notes, files (e.g., medical reports, weather reports, image data, research studies, etc.), electronic text, and/or any data that can be used to determine and/or update the determined wound and/or fall risk. As described above in reference to FIG. 2, in some embodiments, the detailed report 300 (and/or injury detection models) is updated in response to caretaker input.

The relevant analysis section provides a detailed explanation of how the patient's wound and/or fall risk was determined and what features contributed to the determination. The relevant analysis section can indicate factors that improved or worsened a wound and/or fall risk, as well as recommendations on how to improve a determined wound and/or fall risk. The relevant analysis section also includes a user interface element to allow a caretaker to provide additional analysis. The additional analysis can be handwritten notes, files, electronic text, and/or any analysis that can be used to determine and/or update the determined wound and/or fall risk. For example, a caretaker, after meeting with the patient, can provide additional analysis that is used to update a patient's determined wound and/or fall risk (and/or injury detection models) as described above in reference to FIG. 2.

The recommended actions section provides one or more actions that are expected to improve a patient's wound and/or fall risk (e.g., mitigate or prevent a wound and/or fall). In some embodiments, the actions are ranked based on expected contribution to the patient's wound and/or fall risk. For example, recommended actions that are expected to reduce the patient's wound and/or fall risk will be listed first, and recommended actions that do not change the patient's wound and/or fall risk are listed near the bottom. The recommended actions section also includes a user interface element to allow a caretaker to specify additional actions taken. For example, the caretaker can indicate actions that were performed and not listed as a recommended action. Performed recommended actions and/or actions input by the caretaker in the user interface element can update the report (and/or injury detection models) as described above in reference to FIG. 2.

The detailed report 300 can include a risks section that identifies a baseline risk and a current risk for the day. The baseline risk and a current risk for the day can be represented as gauges or meters that fluctuate from a low risk to a high risk based on the patient's wound and/or fall risk (e.g., low risk level (green), low to medium risk level (yellow green), medium risk level (yellow), medium to high risk level (orange), high risk level (red)). In some embodiments, there is a respective baseline risk and a current risk for the day for the wound risk and a respective baseline risk and a current risk for the day for the fall risk. In some embodiments, the baseline risk is based on relatively static patient data. Static patient data includes patient data that does not vary on a daily basis or per each measurement. A non-exhaustive list of static patient data includes chronic medical conditions, diseases, illnesses, age, sex, etc. The current risk for the day is based on dynamic patient data. Alternatively, in some embodiments, the current risk for the day is based on a combination of dynamic patient data and static patient data. Dynamic patient data includes data that can vary on a daily basis or per each measurement. A non-exhaustive list of dynamic patient data includes blood pressure, weight, temperature, diet, skin assessment (e.g., rashes, discoloration, etc.), lung conditions, clinical notes, blood sugar, etc.

The highlights section summarizes one or more features that contributed to the patient's wound and/or fall risk. In some embodiments, the one or more features in the highlights section are ranked in order from features that contributed the most to the patient's wound and/or fall risk to the risk features that contributed the least. The highlights section can include negative and positive features. For example, the highlights section can include both features that improved the wound and/or fall risk and features that worsened the wound and/or fall risk. In some embodiments, the highlights section includes a predetermined number of features (e.g., the top 5, 10, 15, etc.). In this way, the medical practitioner is provided with the top contributing risk features without being overwhelmed by risk features that contributed the least to the patient's wound and/or fall risk. In some implementations, the highlights section includes explanations as to why the feature contributed to the wound and/or fall risk.

As described above in reference to FIG. 2, explanations for wound and/or fall risk and the associated features can be generated by a natural language processing model (e.g., that is part of the report generating module 240). The report generating module 240 can be used to generate human-readable explanations that justify and elaborate on a determined wound and/or fall risk as well as the features that were used to determine the wound and/or fall risk. The explanations generated by the report generating module 240 are configured to provide caretakers with accurate and reliable information that can be used by caretakers to provide efficient and effective care, as well as identify issues or concerns that are not easily discernible by a human (e.g., the patient's diet makes them susceptible to bed sores). The above examples are non-exhaustive and provide a generalized overview of the explanations generated by the report generating module 240.

The diagnosis section, medications section, and orders section user interface elements can present caretakers with the patient's existing diagnosis, medications, and orders. The caretaker can provide an input via the respective section user interface elements (e.g., “Enter Additional . . . ”), and the user inputs can be used to update the injury detection models and/or the detailed report 300.

The progress notes section as shown includes clinical notes for the patient added by a caregiver. In some embodiments, the clinical notes are for a predetermined period of time (e.g., the past seven days, 14 days, 30 days, etc.). The progress notes can include the caretaker's observations in treating a patient, to-do assignments, notes to other caretakers, on-the-fly adjustments to care, and/or other patient information that should be documented to assist in the care of the patient. The progress notes section includes a user interface element that allows the caretakers to include new notes that are used to update the patient's wound risks and/or fall risks and/or the injury detection models (as described above in reference to FIG. 2).

FIG. 4 is a block diagram illustrating a server system 140 in accordance with some embodiments. The server system 140 typically includes one or more central processing units/cores (CPUs) 402, one or more network interfaces 460, memory 406, a power supply 450, and one or more communication buses 408 for interconnecting these components. The communication buses 408 optionally include circuitry (sometimes called a chipset) that interconnects and controls communications between system components. The server system 140 includes I/O interfaces 404. In some embodiments, the I/O interfaces 404 include a keyboard, mouse, microphone, and/or track pad. Alternatively, or in addition, in some embodiments, the I/O interfaces 404 include a display device that includes a touch-sensitive surface, in which case the display device is a touch-sensitive display. “User input,” as described herein, may refer to a contact detected with a touch-sensitive display and/or an input by an I/O interface 404. In some embodiments, the I/O interfaces 404 include a display, speaker, or other devices for providing information (e.g., content, graphs, tables, data, etc.) to a user.

In some embodiments, the one or more network interfaces 460 include wireless and/or wired interfaces for receiving data from and/or transmitting data to one or more healthcare recording databases 110, acute-care facilities 120, post-acute-care facilities 130, and/or other devices or systems. In some embodiments, data communications are carried out using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, in some embodiments, data communications are carried out using any of a variety of custom or standard wired protocols (e.g., USB, Firewire, Ethernet, etc.).

Memory 406 includes high-speed random-access memory, such as DRAM, SRAM, DDR RAM, or other random access solid-state memory devices; and may include non-volatile memory, such as one or more magnetic disk storage devices, optical disk storage devices, flash memory devices, or other non-volatile solid-state storage devices. Memory 406, optionally, includes one or more storage devices remotely located from one or more CPUs 402. Memory 406, or, alternatively, the non-volatile solid-state memory device(s) within memory 406, includes a non-transitory computer-readable storage medium. In some embodiments, memory 406, or the non-transitory computer-readable storage medium of memory 406, stores the following programs, modules and data structures, or a subset or superset thereof:
an operating system 410 that includes procedures for handling various basic system services and for performing hardware-dependent tasks;
a network communication module 412 that is used for connecting the media content server 108 to other computing devices via one or more network interfaces 460 (wired or wireless) connected to one or more networks 170;
one or more server application modules 414 for performing various functions with respect to utilizing machine-learning systems and generating reports identifying patients' risk for returning to an acute-care facility 120, the server application modules 414 including, but not limited to, one or more of:
   a data collecting module 210 for processing historic patient data and determining one or more features to be used in training an injury detection system;
   an injury detection module 420 (analogous to the injury detection system 142) including a training module 220, a detection module 225, and/or a data processing module 235 for training and/or using one or more injury detection modules as described above in reference to FIG. 2;
   a reporting module 430 (analogous to the reporting system 146) including a report generating module 240, a report distribution module 250, and/or a report parsing module 270 for generating, distributing, and/or updating one or more reports, as well as extracting information input into the distributed reports as described above in reference to FIG. 2;
a server database 440 for storing and accessing patient data from the healthcare recording databases 110, acute-care facilities 120, and/or post-acute-care facilities 130, the server database 440 including, but not limited to, one or more of:
   a model database 442 for storing and accessing one or more injury detection models that were previously trained, validated, and tested;
   model data 444 for storing and accessing machine-learning data 215 including training data 216, validation data 217, and test data 218, as well as data processed by the data processing module 235, each of which is used to train and/or use an injury detection model;
   a report database 446 for storing and accessing one or more reports generated by the reporting module 430; and
   patient database 144 for storing and accessing historical patient data 205, current patient data 230, patient data received from healthcare recording databases 110, acute-care facilities 120, and/or post-acute-care facilities 130, and/or any other data described above in reference to FIG. 1.

In some embodiments, the server 140 includes web or Hypertext Transfer Protocol (HTTP) servers, File Transfer Protocol (FTP) servers, as well as web pages and applications implemented using Common Gateway Interface (CGI) script, PHP Hyper-text Preprocessor (PHP), Active Server Pages (ASP), Hyper Text Markup Language (HTML), Extensible Markup Language (XML), Java, JavaScript, Asynchronous Javascript and XML (AJAX), XHP, Javelin, Wireless Universal Resource File (WURFL), and the like.

Each of the above identified modules stored in memory 406 corresponds to a set of instructions for performing a function described herein. The above identified modules or programs (i.e., sets of instructions) need not be implemented as separate software programs, procedures, or modules, and thus various subsets of these modules may be combined or otherwise re-arranged in various embodiments. In some embodiments, memory 406 optionally stores a subset or superset of the respective modules and data structures identified above. Furthermore, memory 406 optionally stores additional modules and data structures not described above. In some embodiments, modules described above with regard to memory 406 are stored at a computer 160 or non-transitory computer system (and vice versa). For example, the reporting module 430 may be stored at the server 140 in memory 406 and/or stored at a computer 160.

Although FIG. 4 illustrates the server 140 in accordance with some embodiments, FIG. 4 is intended as a functional description of the various features that may be present in one or more media content servers than as a structural schematic of the embodiments described herein. In practice, and as recognized by those of ordinary skill in the art, items shown separately could be combined and some items could be separated. For example, some items shown separately in FIG. 4 could be implemented on single servers, and single items could be implemented by one or more servers. The actual number of servers used to implement the server 140, and how features are allocated among them, will vary from one embodiment to another and, optionally, depends in part on the amount of data traffic that the server system handles during peak usage periods as well as during average usage periods.

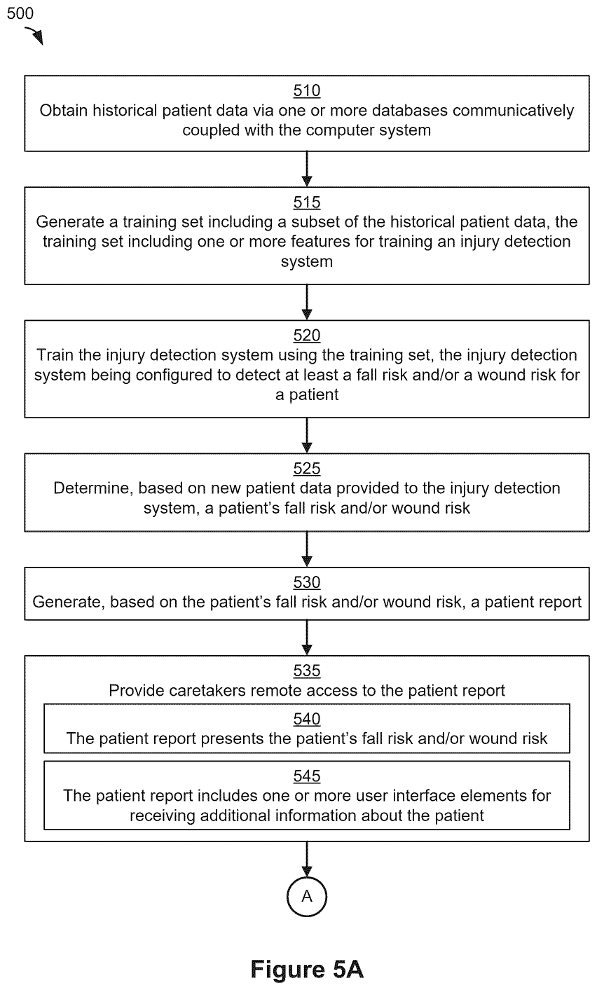

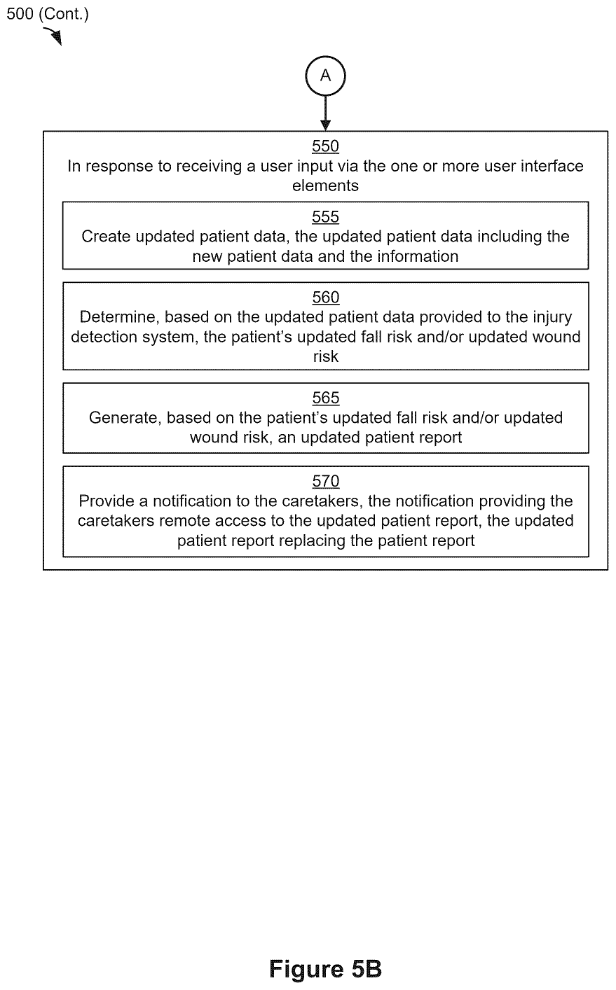

FIGS. 5A and 5B are flow charts illustrating a method 500 of training and using one or more injury detection models for determining wound risks and/or fall risks for patients, in accordance with some embodiments. In some embodiments, method 500 is performed by server 140 (FIGS. 1 and 4). Alternatively, and/or additionally, in some embodiments, method 500 is performed by a computer 160, a mobile device 150, and/or facilities described above in reference to FIG. 1. Operations performed in FIGS. 5A and 5B correspond to instructions stored in computer memory (e.g., memory 406 of server 140, and/or memory of another device). In some embodiments, the methods are performed by a combination of the server 140 and any other device or facility described above in reference to FIG. 1. In some instances and embodiments, the various operations of the methods described herein are interchangeable, and respective operations of the methods are performed by any of the aforementioned devices, systems, or combination of devices and/or systems. For convenience, the method operations will be described below as being performed by a particular component or device, but should not be construed as limiting the performance of the operation to the particular device in all embodiments.

The method 500 is performed at a computer system having one or more processors and memory storing one or more programs that are executable by the computer system.

(A1) The method 500 includes obtaining (510) historical patient data via one or more databases communicatively coupled with the computer system and generating (515) a training set including a subset of the historical patient data. The training set includes one or more features for training an injury detection system. For example, as described above in reference to FIG. 2, the historical patient data 205 is processed by a data collecting module 210 for determining machine-learning data 215 that is used to train one or more injury detection models. The method 500 includes training (520) the injury detection system using the training set. The injury detection system is configured to detect at least a fall risk and/or a wound risk for a patient. As described above in reference to FIG. 2, the training module 220 is used to generate one or more models for determining wound risks and/or fall risks for patients.

The method 500 further includes determining (525), based on new patient data provided to the injury detection system, a patient's fall risk and/or wound risk, generating (530), based on the patient's fall risk and/or wound risk, a patient report, and providing (535) caretakers remote access to the patient report. The patient report presents (540) the patient's fall risk and/or wound risk and includes (545) one or more user interface elements for receiving additional information about the patient. As described above in reference to FIG. 2, the wound risks and/or fall risks determined by the detection module 225 (using trained injury detection models) are used by the report generating module 240 to generate one or more reports that are shared to caretakers 260. The caretakers 260 can provide inputs at the reports, which can be further used to update the determined fall risks and/or wound risks of the patients, the current patient data 230, and/or the historical patient data 205. Example reports generated by the report generating module 240 are shown in FIGS. 3A and 3B.

The method 500 further includes, in response to receiving (550) a user input via the one or more user interface elements (of a generated report), creating (555) updated patient data and determining (560), based on the updated patient data provided to the injury detection system, the patient's updated fall risk and/or updated wound risk. The updated patient data includes the new patient data and the information. The method 500 also includes generating (565), based on the patient's updated fall risk and/or updated wound risk, an updated patient report, and providing (570) a notification to the caretakers. The notification provides the caretakers remote access to the updated patient report. In some embodiments, the updated patient report replaces the patient report. For example, as described above in reference to FIG. 2, the caretakers 260 can provide one or more inputs at a report distributed by the report distribution module 250, the inputs provided by the caretakers 260 can be processed (e.g., by a report parsing module 270) and be used to notify caretakers 260 of an update, update the provided reports, update the current patient data 230, and/or update the historical patient data 205.

(A2) In some embodiments of A1, the new patient data includes respective patient data for a plurality of patients; a respective patient's fall risk and/or wound risk is determined for each patient of the plurality of patients using the injury detection system and based on the respective patient data for the plurality of patients; and a respective patient report is generated for each patient of the plurality of patients. For example, as described above in reference to FIGS. 2-3B, a report can be generated for each patient, and each patient can be listed based on their respective risks.

(A3) In some embodiments of any one of A1-A2, a portion of the information is in an unstructured format (e.g., handwritten notes). In some embodiments, another portion of the information is in a structured format (e.g., inputs in a drop-down menu, radio button, etc.).

(A4) In some embodiments of any one of A1-A3, the method 500 further includes determining, using the injury detection system, a human-readable explanation for the patient's fall risk and determining, using the injury detection system, a human-readable explanation for the patient's wound risk. The patient report includes the human-readable explanation for the patient's fall risk and the human-readable explanation for the patient's wound risk. For example, as described above in reference to FIG. 2, the reporting system 146 can be used to generate one or more reports that are used to explain to a caretaker justification of a particular risk as well as factors (e.g., features) that contributed to the respective risk.

(A5) In some embodiments of any one of A1-A4, the method 500 further includes determining, using the injury detection system, a first plurality of factors contributing to the patient's fall risk and determining, using the injury detection system, a second plurality of factors contributing to the patient's wound risk. The patient report includes the first plurality of factors and the second plurality of factors. As described above in reference to FIG. 2, the reporting system 146 can be used to generate one or more reports that identify factors (e.g., features) that contributed to a respective risk.

(A6) In some embodiments of A5, factors of the first plurality of factors and the second plurality of factors are ranked from highest contributing factor to least contributing factor. As described above in reference to FIG. 2, individual features can be listed and/or weighted based on their contribution to a particular risk score.

(A7) In some embodiments of any one of A1-A6, the method 500 further includes determining, using the injury detection system, one or more first mitigating suggestions for ameliorating the patient's fall risk and determining, using the injury detection system, one or more second mitigating suggestions for ameliorating the patient's wound risk. The patient report includes the first mitigating suggestions and the second mitigating suggestions for the patient's fall risk, respectively. As described above in reference to FIG. 2, the reporting system 146 can be used to generate one or more reports that identify mitigating factors (e.g., mitigating features) that can change a respective risk.

(A8) In some embodiments of A7, mitigating suggestions of the first mitigating suggestions and the second mitigating suggestions are ranked from highest contributing factor to least contributing factor. As described above in reference to FIG. 2, individual features can be listed and/or weighted based on their contribution to a particular risk score.

(A9) In some embodiments of any one of A1-A8, the method 500 further includes, in response to obtaining updated historical patient data via the one or more databases communicatively coupled with the computer system, generating an updated training set including an updated subset of the historical patient data, training the updated injury detection system using the updated training set, and replacing the injury detection system with the updated injury detection system. As described above in reference to FIG. 2, either the current patient data 230, the historical patient data 205, or both can be updated and used to update the respective risks and/or injury detection models.

(A10) In some embodiments of A9, the updated historical patient data is received periodically.

(A11) In some embodiments of any one of A1-A10, the patient report includes a patient rank indicating the patient's respective fall risk and/or wound risk in relation to other patients.

(A12) In some embodiments of any one of A1-A11, the method 500 further includes, in response to detection of a triggering event associated with a respective patient, providing another notification to the caretakers, the other notification providing the caretakers remote access to a respective patient report.

(A13) In some embodiments of A12, the triggering event includes a fall risk above a predetermined fall risk threshold, a wound risk above a predetermined wound risk threshold, and an update to a patient report. Examples of the different triggering events are described above in reference to FIG. 2.

(B1) In accordance with some embodiments, a non-transitory computer readable storage medium including instructions that, when executed by a computer device, cause the computer device to perform operations corresponding to any of A1-13.

(C1) In accordance with some embodiments, a system including an electronic device communicatively coupled with at least a patient data database, the system configured to perform or cause performance of the operations that correspond to any of A1-A13.

(D1) In accordance with some embodiments, a means for performing or causing performance of the operations corresponding to any of A1-A13.

(E1) In accordance with some embodiments, an electronic device configured to perform or cause the performance of the operations corresponding to any of A1-A13.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles and their practical applications, to thereby enable others skilled in the art to best utilize the embodiments and various embodiments with various modifications as are suited to the particular use contemplated.

It will also be understood that, although the terms “first,” “second,” etc. are, in some instances, used herein to describe various elements, these elements should not be limited by these terms. These terms are used only to distinguish one element from another. For example, a first client device could be termed a second client device, and, similarly, a second client device could be termed a first client device, without departing from the scope of the various described embodiments. The first client device and the second client device are both client devices, but they are not the same client device.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

What is claimed is:

1. A method of notifying caretakers of patient's risk of falling or incurring a wound, the method comprising:
at a computer system having one or more processors and memory storing one or more programs that are executable by the computer system:
obtaining historical patient data via one or more databases communicatively coupled with the computer system;
generating a training set including a subset of the historical patient data, the training set including one or more features for training an injury detection system;
training the injury detection system using the training set, wherein the injury detection system is configured to detect at least a fall risk and/or a wound risk for a patient;
determining, based on new patient data provided to the injury detection system, a patient's fall risk and/or wound risk;
generating, based on the patient's fall risk and/or wound risk, a patient report;
providing caretakers remote access to the patient report, wherein the patient report:
presents the patient's fall risk and/or wound risk, and
includes one or more user interface elements for receiving additional information about the patient; and
in response to receiving a user input via the one or more user interface elements:
creating updated patient data, the updated patient data including the new patient data and the information,
determining, based on the updated patient data provided to the injury detection system, the patient's updated fall risk and/or updated wound risk,
generating, based on the patient's updated fall risk and/or updated wound risk, an updated patient report, and
providing a notification to the caretakers, the notification providing the caretakers remote access to the updated patient report, wherein the updated patient report replaces the patient report.

2. The method of claim 1, wherein:
the new patient data includes respective patient data for a plurality of patients;
a respective patient's fall risk and/or wound risk is determined for each patient of the plurality of patients using the injury detection system and based on the respective patient data for the plurality of patients; and
a respective patient report is generated for each patient of the plurality of patients.

3. The method of claim 1, further comprising:
determining, using the injury detection system, a human-readable explanation for the patient's fall risk;
determining, using the injury detection system, a human-readable explanation for the patient's wound risk; and
wherein the patient report includes the human-readable explanation for the patient's fall risk and the human-readable explanation for the patient's wound risk.

4. The method of claim 1, further comprising:
determining, using the injury detection system, a first plurality of factors contributing to the patient's fall risk;
determining, using the injury detection system, a second plurality of factors contributing to the patient's wound risk; and
wherein the patient report includes the first plurality of factors and the second plurality of factors.

5. The method of claim 4, wherein factors of the first plurality of factors and the second plurality of factors are ranked from highest contributing factor to least contributing factor.

6. The method of claim 1, further comprising:
determining, using the injury detection system, one or more first mitigating suggestions for ameliorating the patient's fall risk;
determining, using the injury detection system, one or more second mitigating suggestions for ameliorating the patient's wound risk; and
wherein the patient report includes the first mitigating suggestions and the second mitigating suggestions for the patient's fall risk, respectively.

7. The method of claim 6, wherein mitigating suggestions of the first mitigating suggestions and the second mitigating suggestions are ranked from highest contributing factor to least contributing factor.

8. The method of claim 1, further comprising:
in response to obtaining updated historical patient data via the one or more databases communicatively coupled with the computer system:
generating an updated training set including an updated subset of the historical patient data, the updated training set including the one or more features for training an updated injury detection system;
training the updated injury detection system using the updated training set; and
replacing the injury detection system with the updated injury detection system.

9. The method of claim 1, wherein the patient report includes a patient rank indicating the patient's respective fall risk and/or wound risk in relation to other patients.

10. The method of claim 1, further comprising:
in response to detection of a triggering event associated with a respective patient, providing another notification to the caretakers, the other notification providing the caretakers remote access to a respective patient report.

11. The method of claim 10, wherein the triggering event includes a fall risk above a predetermined fall risk threshold, a wound risk above a predetermined wound risk threshold, and an update to a patient report.

12. A system, comprising:
a memory storing instructions; and
one or more processors coupled to the memory resource, the one or more processors being configured to execute the instructions to:
obtain historical patient data via one or more databases communicatively coupled with the system;
generate a training set including a subset of the historical patient data, the training set including one or more features for training an injury detection system;
train the injury detection system using the training set, wherein the injury detection system is configured to detect at least a fall risk and/or a wound risk for a patient;
determine, based on new patient data provided to the injury detection system, a patient's fall risk and/or wound risk;
generate, based on the patient's fall risk and/or wound risk, a patient report;
provide caretakers remote access to the patient report, wherein the patient report:
presents the patient's fall risk and/or wound risk, and
includes one or more user interface elements for receiving additional information about the patient; and
in response to receiving a user input via the one or more user interface elements:
create updated patient data, the updated patient data including the new patient data and the information,
determine, based on the updated patient data provided to the injury detection system, the patient's updated fall risk and/or updated wound risk,
generate, based on the patient's updated fall risk and/or updated wound risk, an updated patient report, and
provide a notification to the caretakers, the notification providing the caretakers remote access to the updated patient report, wherein the updated patient report replaces the patient report.

13. The system of claim 12, wherein:
the new patient data includes respective patient data for a plurality of patients;
a respective patient's fall risk and/or wound risk is determined for each patient of the plurality of patients using the injury detection system and based on the respective patient data for the plurality of patients; and
a respective patient report is generated for each patient of the plurality of patients.

14. The system of claim 12, wherein the instructions, when executed by the one or more processors, further cause the one or more processors to:
determine, using the injury detection system, a human-readable explanation for the patient's fall risk;
determine, using the injury detection system, a human-readable explanation for the patient's wound risk; and
wherein the patient report includes the human-readable explanation for the patient's fall risk and the human-readable explanation for the patient's wound risk.

15. The system of claim 12, wherein the instructions, when executed by the one or more processors, further cause the one or more processors to:
determine, using the injury detection system, a first plurality of factors contributing to the patient's fall risk;
determine, using the injury detection system, a second plurality of factors contributing to the patient's wound risk; and
wherein the patient report includes the first plurality of factors and the second plurality of factors.

16. The system of claim 15, wherein factors of the first plurality of factors and the second plurality of factors are ranked from highest contributing factor to least contributing factor.

17. A non-transitory computer-readable storage medium storing instructions that when executed by a processor, causes the processor to:
obtain historical patient data via one or more databases communicatively coupled with a computer system;
generate a training set including a subset of the historical patient data, the training set including one or more features for training an injury detection system;
train the injury detection system using the training set, wherein the injury detection system is configured to detect at least a fall risk and/or a wound risk for a patient;
determine, based on new patient data provided to the injury detection system, a patient's fall risk and/or wound risk;
generate, based on the patient's fall risk and/or wound risk, a patient report;
provide caretakers remote access to the patient report, wherein the patient report:
presents the patient's fall risk and/or wound risk, and
includes one or more user interface elements for receiving additional information about the patient; and
in response to receiving a user input via the one or more user interface elements:
create updated patient data, the updated patient data including the new patient data and the information,
determine, based on the updated patient data provided to the injury detection system, the patient's updated fall risk and/or updated wound risk,
generate, based on the patient's updated fall risk and/or updated wound risk, an updated patient report, and
provide a notification to the caretakers, the notification providing the caretakers remote access to the updated patient report, wherein the updated patient report replaces the patient report.

18. The non-transitory computer-readable storage medium of claim 17, wherein:
the new patient data includes respective patient data for a plurality of patients;
a respective patient's fall risk and/or wound risk is determined for each patient of the plurality of patients using the injury detection system and based on the respective patient data for the plurality of patients; and
a respective patient report is generated for each patient of the plurality of patients.

19. The non-transitory computer-readable storage medium of claim 17, wherein the instructions, when executed by the processor, further cause the processor to:
determine, using the injury detection system, a human-readable explanation for the patient's fall risk;
determine, using the injury detection system, a human-readable explanation for the patient's wound risk; and
wherein the patient report includes the human-readable explanation for the patient's fall risk and the human-readable explanation for the patient's wound risk.

20. The non-transitory computer-readable storage medium of claim 17, wherein the instructions, when executed by the processor, further cause the processor to:
determine, using the injury detection system, a first plurality of factors contributing to the patient's fall risk;
determine, using the injury detection system, a second plurality of factors contributing to the patient's wound risk; and
wherein the patient report includes the first plurality of factors and the second plurality of factors.
